(19) United States
(12) Patent Application Publication
(10) Pub. No.: US 2024/0287499 A1
(43) Pub. Date: Aug. 29, 2024

(54) CARTRIDGE SYSTEM FOR EXTRACTION OF COMPLEX SAMPLE, AND COMPLEX SAMPLE EXTRACTING METHOD

(71) Applicant: MICRO DIGITAL CO., LTD, Seongnam-si, Gyeonggi-do (KR)

(72) Inventors: Kyung Nam KIM, Gwangju-si, Gyeonggi-do (KR); Se Hak LIM, Gunpo-si, Gyeonggi-do (KR); Jong In RYU, Yongin-si, Gyeonggi-do (KR)

(21) Appl. No.: 18/573,620

(22) PCT Filed: Jul. 1, 2022
(86) PCT No.: PCT/KR2022/009509

§ 371 Date: Dec. 22, 2023

(30) Foreign Application Priority Data
Jul. 16, 2021 (KR) 10-2021-0093378

(51) Int. Cl. C12N 15/10; G01N 35/00; G01N 35/10

(52) U.S. Cl. CPC C12N 15/1013; C12N 15/1017; G01N 35/0099; G01N 35/10; G01N 2035/00465

(57) ABSTRACT

The cartridge system for extraction of a complex sample may include: a cartridge body; a complex sample collector for collecting a complex sample to provide same to the cartridge body; a buffer solution receiving unit formed in the cartridge body and configured to accommodate a buffer solution therein and provide a space in which, after a collecting unit of the complex sample collector is immersed in the buffer solution, the collecting unit is rotated by a spin head of a robot device such that the complex sample is mixed with the buffer solution or mixed beads included in the buffer solution; and a complex sample collector disposal unit formed in the cartridge body and configured to temporarily store the complex sample collector to discard same after the complex sample is provided to the buffer solution receiving unit.

### TECHNICAL FIELD

The present invention relates to a cartridge system for extraction of a complex sample and a complex sample extracting method, and more specifically, to a cartridge system for extraction of a complex sample and a complex sample extracting method which allow a DNA or RNA component to be extracted from a complex sample such as stool with a mixture of liquid and solid phases through a series of pretreatment processes.

### BACKGROUND ART

Molecular diagnosis refers to a diagnostic method that directly analyzes the genes (DNA or RNA) of a target substance in a sample to identify the presence of infection of disease, base sequence variations, or mutations, enabling early disease diagnosis and efficient treatment.

Recently, molecular diagnostic methods have been widely used in various medical fields, including confirmation of disease infection, genetic testing, and pharmacogenetic testing.

Various detection methods have been developed for the molecular diagnostic methods, with real-time polymerase chain reaction (PCR) becoming widely used due to its speed, convenience, and sensitivity in detection. Real-time PCR typically uses a probe that forms a specific complementary binding with the gene of a target substance, and fluorescence molecules are attached to the probe. In real-time PCR, the wavelength of these fluorescence molecules is analyzed by an analyzing device to qualitatively/quantitatively analyze the target gene.

In the molecular diagnostic methods, through real-time PCR, the target substance collected on a swab or a collection part is subjected to pretreatment before analysis, and the pretreated substance, i.e., a buffer solution, is analyzed. In conventional techniques, to collect specimens using swabs, a target specimen, i.e., sample, may be collected in various ways, for example, by using nasal swabs, nasopharyngeal swabs, throat swabs, and the like.

However, the molecular diagnostic methods using swabs are limited to collecting specimens in a liquid state, and thus unable to test complex samples where liquid and solid are mixed, such as stool. Particularly, to collect the specimen on the swab, it is necessary to insert the swab deep into the area, such as the nasal cavity, nasopharynx, or throat, which causes discomfort to an examination subject, leading to significant resistance, or the collection process is not smooth, which, in turn, results in decreased precision of test results. Additionally, there are many side effects, such as inducing coughing in the examination subject, potentially spreading bacteria or viruses to the collector.

### DETAILED DESCRIPTION OF THE INVENTION

### Technical Problem

The present invention is to provide a cartridge system for extraction of a complex sample and a complex sample extracting method that enable molecular diagnostic testing of a complex sample with a mixture of liquid and solid phases, such as the stool of an examination subject, preventing physical discomfort or resistance in the examination subject, facilitating sample collection to significantly enhance the precision of test results, and minimizing the spread of bacteria or viruses during the sample collection process. However, the above object is illustrative only, and does not limit the scope of the present invention.

### Technical Solution

According to an aspect of the present invention, there is provided a cartridge system for extraction of a complex sample, and a complex sample extracting method, wherein a DNA or RNA component can be extracted from a complex sample such as feces with a mixture of liquid and solid phases through a series of pretreatment processes. The cartridge system for extraction of a complex sample may comprise: a cartridge body; a complex sample collector for collecting a complex sample to provide same to the cartridge body; a buffer solution receiving unit formed in the cartridge body and configured to accommodate a buffer solution therein and provide a space in which, after a collecting unit of the complex sample collector is immersed in the buffer solution, the collecting unit is rotated by a spin head of a robot device such that the complex sample is mixed with the buffer solution or mixed beads included in the buffer solution; and a complex sample collector disposal unit formed in the cartridge body and configured to temporarily store the complex sample collector to discard same after the complex sample is provided to the buffer solution receiving unit.

In addition, according to the present invention, the complex sample collector may include: a collector body having an overall shape of a pipe and a hollow portion formed therein; and a collection plunger installed to move up and down in the hollow portion, exposing the collecting unit when the button unit is pressed, and sealing the collected complex sample by retracting the collecting unit into the collector body when the button unit is raised after collecting the complex sample.

In addition, according to the present invention, the complex sample collector may further include an isolation protrusion unit formed on a side surface of the collection plunger and spaced apart from the collector body to facilitate rotation of the collection plunger when the button unit descends; and a forced-engagement protrusion unit formed on a side surface of the collection plunger to be forcibly engaged with the collector body such that the collecting unit is sealed when the button unit rises.

In addition, according to the present invention, the complex sample collector may further include a locking flange unit installed at an entrance of the collector body and shaped to correspond to a height-lock slot formed on an upper surface of the buffer solution receiving unit such that the spin head of the robot device can be inserted in the direction of the height-lock slot and locked into the height-lock slot.

In addition, according to the present invention, the cartridge system may further include: a first tip receiving unit formed in the cartridge body and configured to accommodate the first tip such that a first tip is attached to a pump head of the robot device; and a filter unit receiving unit formed in the cartridge body and configured to accommodate a filter unit such that the pump head of the robot device attaches the filter unit to the first tip and primarily filters the buffer solution accommodated in the buffer solution receiving unit through the filter unit.

In addition, according to the present invention, the cartridge system may further include a second tip receiving unit formed in the cartridge body and configured to accommodate a second tip such that the pump head of the robot device uses the first tip to aspirate the buffer solution and attaches the second tip to the first tip.

Additionally, according to the present invention, the filter unit may include a filter unit body forcibly engaged with the first tip; and a flange unit formed on a bottom of the filter unit body and having a plurality of filter holes formed to primarily filter the buffer solution.

In addition, according to the present invention, the first tip may include a first forced-engaging unit formed to forcibly engage with the filter unit, the buffer solution receiving unit may include a second forced-engaging step unit formed to forcibly engage with the filter unit, and a first engagement strength of the first forced-engaging unit may be lower than a second engagement strength of the second forced-engaging step unit such that, after the primary filtering by the filter unit, the filter unit remains in the buffer solution receiving unit, and instead, the first tip is easily separated from the filter unit.

Additionally, according to the present invention, the second tip may further include: a second tip body forcibly engaged with the first tip; and a secondary filter installed inside the second tip body and including a mesh filter or a membrane filter.

In addition, according to the present invention, the cartridge system may further include a sample solution receiving unit formed in the cartridge body and configured to accommodate a sample solution that the pump head of the robot device dispenses while secondarily filtering the buffer solution using the second tip; a first precision dispensing tip receiving unit formed in the cartridge body and configured to accommodate first precision dispensing tip such that the pump head of the robot device is coupled with the first precision dispensing tip to aspirate the sample solution; a first washing chamber unit formed in the cartridge body and configured to accommodate a first washing solution and magnetic beads included in the first washing solution such that the sample solution aspirated by the pump head of the robot device is mixed with the first washing solution and the magnetic beads to retain only DNA components on the magnetic beads; a stirring tip receiving unit formed in the cartridge body and configured to accommodate a stirring tip such that the spin head of the robot device is coupled with the stirring tip and stirs the magnetic beads in the first washing chamber unit with the first washing solution while rotating or moving up and down; and an Nth washing chamber unit (N is a natural number greater than or equal to 2) formed in the cartridge body and configured to accommodate an Nth washing solution such that, as the stirring tip of the spin head of the robot device rotates or moves up and down, the magnetic beads are mixed with the Nth washing solution, allowing only DNA components to remain.

In addition, according to the present invention, the stirring tip may include: a rotatable stirring tip body having an overall hollow shape and coupled with the spin head; and a magnetic bar installed inside the stirring tip body and magnetically attaching the magnetic beads to the stirring tip body while moving up and down.

Additionally, according to the present invention, the cartridge system may include: an elution chamber unit formed in the cartridge body and configured to accommodate an elution solution such that the DNA components remaining on the magnetic bead are separated into the elution solution as the stirring tip of the spin head of the robot device rotates or moves up and down; a second precision dispensing tip receiving unit formed in the cartridge body and configured to accommodate a second precision dispensing tip such that the pump head of the robot device attaches to the second precision dispensing tip; and a pipetting chamber unit formed in the cartridge body and configured to accommodate the elution solution dispensed such that the pump head of the robot device aspirates the elution solution using the second precision dispensing tip and dispenses the aspirated elution solution into the pipetting chamber unit.

According to another aspect of the present invention, there is provided a complex sample extracting method including: (a) immersing a collecting unit of a complex sample collector, which collects a complex sample, into a buffer solution accommodated in a buffer solution receiving unit and mixing the complex sample with the buffer solution or mixed beads included in the buffer solution by rotating the collecting unit using a spin head of a robot device; (b) attaching a first tip to a pump head of the robot device, attaching a filter unit to the first tip, and primarily filtering the buffer solution accommodated inside the buffer solution receiving unit through the filter unit; (c) at the pump head of the robot device, aspirating the buffer solution using the first tip and attaching a second tip to the first tip; and (d) receiving a sample solution that the pump head of the robot device dispenses while secondarily filtering the buffer solution accommodated in the first tip using the second tip.

In addition, according to the present invention, operation (b) may include: (b-1) forcibly engaging the first tip with the filter unit; (b-2) inserting the filter unit into the buffer solution receiving unit; and (b-3) primarily filtering the buffer solution by pressing the buffer solution mixed with the complex sample using the filter unit.

Additionally, according to the present invention, operation (c) may include: (c-1) at the pump head of the robot device separated from the filter unit, aspirating the buffer solution using the first tip; (c-2) raising the pump head of the robot device to separate the first tip from the filter unit while being attached to the pump head of the robot device while the filter unit remains in the buffer solution receiving unit; and (c-3) at the pump head of the robot device separated from the filter unit, attaching the second tip to the first tip.

In addition, according to the present invention, the complex sample extracting method may further include: (e) coupling a first precision dispensing tip to the pump head of the robot device and aspirating the sample solution; (f) mixing the sample solution aspirated by the pump head of the robot device with a first washing solution accommodated in a first washing chamber unit and magnetic beads included in the first washing solution, allowing only DNA components to remain on the magnetic beads; (g) coupling a stirring tip to the spin head of the robot device and rotating or moving up and down the stirring tip to mix the magnetic beads in the first washing chamber unit with the first washing solution, allowing only the DNA components to remain; (i) separating the DNA components remaining on the magnetic beads into an elution solution while rotating or moving up and down the stirring tip of the spin head of the robot device; and (j) coupling a second precision dispensing tip to the pump head of the robot device, aspirating the elution solution using the second precision dispensing tip, and dispensing the aspirated elution solution into a pipetting chamber unit.

In addition, according to the present invention, the complex sample extracting method may further include, before or after operation (i), (h) mixing the magnetic beads with an Nth washing solution (N is a natural number greater than or equal to 2) while rotating or moving up and down the stirring tip of the spin head of the robot device, allowing only the DNA components to remain.

### Effect of the Invention

According to some embodiments of the present invention as described above, it is possible to conduct molecular diagnostic testing on a complex sample, such as stool containing a mixture of liquid and solid phases, thereby preventing physical discomfort or resistance in an examination subject, facilitating sample collection to significantly enhance the precision of test results, and minimizing the transmission of bacteria or viruses during the sample collection process. However, the above effects do not limit the scope of the present invention.

### BRIEF DESCRIPTION OF DRAWINGS

FIG. 1 is an exterior perspective view of a cartridge system for extraction of a complex sample according to some embodiments of the present invention.

FIG. 2 is a plan view of the cartridge system for extraction of a complex sample of FIG. 1.

FIG. 3 is a cross-sectional view of the cartridge system for extraction of a complex sample of FIG. 1.

FIGS. 4 to 22 are cross-sectional views of stages of a sample extraction process of the cartridge system for extraction of a complex sample of FIG. 1.

FIG. 23 is a flowchart illustrating a complex sample extracting method according to some embodiments of the present invention.

FIG. 24 is a flowchart illustrating in more detail operation (b) of the complex sample extracting method of FIG. 23.

FIG. 25 is a flowchart illustrating in more detail operation (c) of the complex sample extracting method of FIG. 23.

### MODE FOR INVENTION

Hereinafter, exemplary embodiments of the present invention will be described in detail with reference to the accompanying drawings.

The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. In the drawings, the thicknesses or sizes of layers are exaggerated for clarity and convenience of explanation.

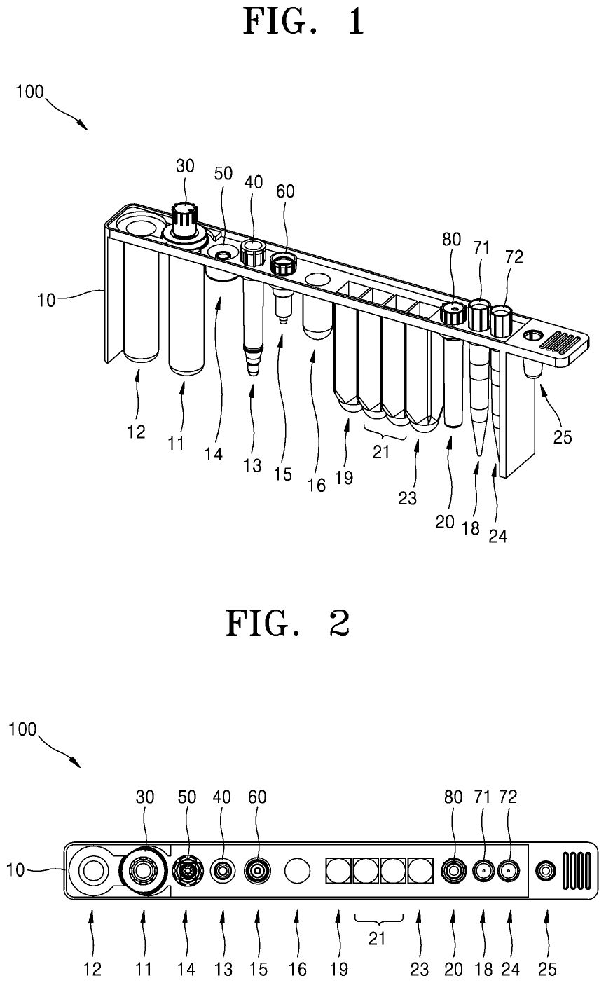

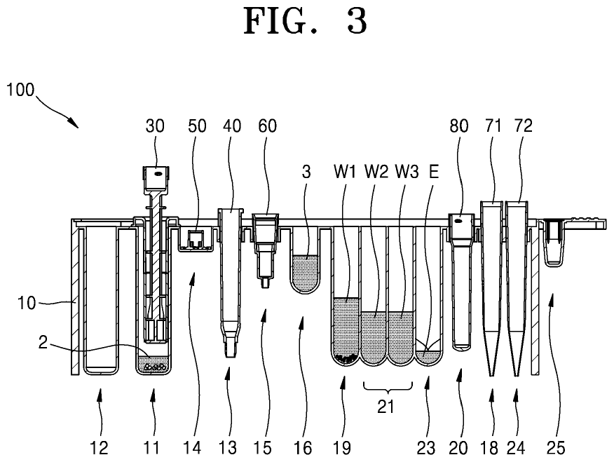

FIG. 1 is an exterior perspective view of a cartridge system 100 for extraction of a complex sample according to some embodiments of the present invention, FIG. 2 is a plan view of the cartridge system 100 for extraction of a complex sample of FIG. 1, and FIG. 3 is a cross-sectional view of the cartridge system 100 for extraction of a complex sample of FIG. 1.

First, as shown in FIGS. 1 to 3, the cartridge system 100 for extraction of a complex sample according to some embodiments of the present invention may largely include a cartridge body 10, a buffer solution receiving unit 11 formed in the cartridge body 10, a complex sample collector disposal unit 12, a first tip receiving unit 13, a filter unit receiving unit 14, a second tip receiving unit 15, a sample solution receiving unit 16, a first precision dispensing tip receiving unit 18, a first washing chamber unit 19, a stirring tip receiving unit 20, an Nth washing chamber unit 21, an elution chamber unit 23, a second precision dispensing tip receiving unit 24, and a pipetting chamber unit 25. Further, the cartridge system 100 for extraction of a complex sample may further include a complex sample collector 30 configured to collect a complex sample and provide it to the cartridge body 10.

For example, as shown in FIGS. 1 to 3, the cartridge body 10, which may be detachably mounted inside inspection equipment (not shown) installed at an inspection site or at the point of care, may be an integrated structure made of synthetic resin material or metal material with sufficient strength and durability to support the buffer solution receiving unit 11, the complex sample collector disposal unit 12, the first tip receiving unit 13, the filter unit receiving unit 14, the second tip receiving unit 15, the sample solution receiving unit 16, the first precision dispensing tip receiving unit 18, the first washing chamber unit 19, the stirring tip receiving unit 20, the Nth washing chamber unit 21, the elution chamber unit 23, the second precision dispensing tip receiving unit 24, and the pipetting chamber unit 25 described above.

However, the shape, type, material, design, and the like of the cartridge body 10 are not limited thereto, and they may be modified or changed as needed depending on the specifications of the equipment where the cartridge system is to be mounted, the inspection environment, or the required specifications.

More specifically, as shown in FIGS. 1 to 3, for example, the buffer solution receiving unit 11, formed in the cartridge body 10, may serve as a part that may accommodate a buffer solution 2 therein, provide a space in which, after a collecting unit 32b of the complex sample collector 30 is immersed in the buffer solution 2, the collecting unit is rotated by a spin head SH of a robot device such that the complex sample 1 is mixed with the buffer solution 2 or mixed beads B1 included in the buffer solution 2, and accommodate both the buffer solution 2 and the complex sample collector 30.

Additionally, for example, as shown in FIGS. 1 to 3, the complex sample collector disposal unit 12, formed in the cartridge body 10, may serve as a part that temporarily stores the complex sample collector 30 to discard the same after the complex sample 1 is to the buffer solution receiving unit 11.

Additionally, for example, as shown in FIGS. 1 to 3, the first tip receiving unit 13, formed in the cartridge body 10, may serve as a part that accommodates the first tip 40 such that the pump head PH of the robot device attaches the first tip 40 thereto. Also, for example, as shown in FIGS. 1 to 3, the filter unit receiving unit 14, formed in the cartridge body 10, may serve as a part that accommodates the filter unit 50 such that the pump head PH of the robot device attaches the filter unit 50 to the first tip 40 and primarily filters the buffer solution 2, for example, fibers or debris contained in the buffer solution 2, within the buffer solution receiving unit 11 using the filter unit 50. These fibers or debris may originate from the provided complex sample 1.

In addition, for example, as shown in FIGS. 1 to 3, the second tip receiving unit 15, formed in the cartridge body 10, may serve as a part that accommodates the second tip 60 such that the pump head PH of the robot device uses the first tip 40 to aspirate the buffer solution 2 and attaches the second tip 60 to the first tip 40. Specifically, while the first tip 40 aspirates and holds the buffer solution 2, the second tip 60 may be attached to one end of the first tip 40.

Moreover, for example, as shown in FIGS. 1 to 3, the sample solution receiving unit 16, formed in the cartridge body 10, may serve as a part that accommodates the sample solution 3 that is dispensed by the pump head PH of the robot device while secondarily filtering the buffer solution 2 using the second tip 60.

The sample solution 3 may refer to substances formed after the mixture of the complex sample 1 and the buffer solution 2 is filtered. Therefore, the sample solution 3 may refer to substances including the buffer solution 2, the liquid substance of the complex sample 1, and the liquid substance formed by dissolving the solid substance of the complex sample 1 in the buffer solution 2.

After dispensing the sample solution 3 into the sample solution receiving unit 16, the buffer solution receiving unit 11, the first tip receiving unit 13, or the second tip receiving unit 15 temporarily stores the engaged first tip 40 and second tip 60, functioning as a first and second tip disposal unit that discards the engaged first tip 40 and the second tip 60. Additionally, the first and second tip disposal unit may be provided as a separate configuration.

In addition, for example, as shown in FIGS. 1 to 3, the first precision dispensing tip receiving unit 18, formed in the cartridge body 10, may serve as a part that accommodates the first precision dispensing tip 71 such that the pump head PH of the robot device is coupled with the first precision dispensing tip 71 to aspirate the sample solution 3.

Also, for example, as shown in FIGS. 1 to 3, the first washing chamber unit 19, formed in the cartridge body 10, may serve as a part that accommodates a first washing solution W1 and magnetic beads B2 such that the sample solution 3 aspirated by the pump head PH of the robot device is mixed with the first washing solution W1 and the magnetic beads B2 included in the first washing solution W1, allowing only DNA components to remain on the magnetic beads B2.

The magnetic beads B2 may consist of a material with magnetic properties, for example, metal. The magnetic beads B2 may be composed of various materials, shapes, and forms to which the DNA components can be attached physically, chemically, biologically, etc.

In addition, for example, as shown in FIGS. 1 to 3, the stirring tip receiving unit 20, formed in the cartridge body 10, may serve as a part that accommodates the stirring tip 80 such that the spin head SH of the robot device is coupled with the stirring tip 80 and stirs the magnetic beads B2 with the first washing solution W1 in the first washing chamber unit 19 while rotating or moving up and down.

Additionally, the stirring tip receiving unit 20 may function as a stirring tip disposal unit that temporarily stores the stirring tip 80 for subsequent disposal of the stirring tip 80. Also, the agitator tip disposal unit may be provided as a separate configuration.

Moreover, for example, as shown in FIGS. 1 to 3, the Nth washing chamber unit 21, formed in the cartridge body 10, may serve as a part that accommodates an Nth washing solution WN (where N is a natural number greater than or equal to 2) such that as the stirring tip 80 of the spin head SH of the robot device rotates or moves up and down, the magnetic beads B2 magnetically attached to and transferred by the stirring tip 80 are mixed with the Nth washing solution WN, allowing only the DNA components to remain.

Additionally, for example, as shown in FIGS. 1 to 3, the elution chamber unit 23, formed in the cartridge body 10, may serve as a part that accommodates an elution solution E such that the DNA components remaining on the magnetic beads B2 magnetically attached to and transferred by the stirring tip 80 of the spin head SH of the robot device are separated into the elution solution E as the stirring tip 80 rotates or moves up and down.

Also, for example, as shown in FIGS. 1 to 3, the second precision dispensing tip receiving unit 24, formed in the cartridge body 10, may serve as a part that accommodates the second precision dispensing tip 72 such that the pump head PH of the robot device is coupled with the second precision dispensing tip 72.

Moreover, for example, as shown in FIGS. 1 to 3, the pipetting chamber unit 25, formed in the cartridge body 10, may serve as a part that accommodates the elution solution E dispensed such that the pump head PH of the robot device aspirates the elution solution E using the second precision dispensing tip 72 and dispenses the aspirated elution solution E into the pipetting chamber unit 25.

Here, for example, as shown in FIGS. 1 to 3, according to the order of the pretreatment processes for extraction of a complex sample, the buffer solution receiving unit 11, the complex sample collector disposal unit 12, the first tip receiving unit 13, the filter unit receiving unit 14, the second tip receiving unit 15, the sample solution receiving unit 16, the first and second tip disposal unit 17, the first precision dispensing tip receiving unit 18, the first washing chamber unit 19, the stirring tip receiving unit 20, the Nth washing chamber unit 21, the elution chamber unit 23, the second precision dispensing tip receiving unit 24, and the pipetting chamber unit 25 may be arranged in a line in this order, from the front end to the rear end of the upper surface of the cartridge body 10.

However, this order is not necessarily limited to the drawings, and various rearrangements are possible as needed.

Therefore, as shown in FIGS. 1 to 3, the moving distance of the spin head SH and pump head PH of the robot device installed in the aforementioned inspection equipment may be minimized. In other words, while the spin head SH and pump head PH intermittently move from the front end to the rear end of the cartridge body 10, a series of sample extraction pretreatment processes of extracting the complex sample 1 from the complex sample collector 30 and dispersing the same into the elution solution E in the pipetting chamber unit 25 may be sufficiently performed sequentially within only a single cartridge system for extraction of a complex sample according to some embodiments of the present invention.

Therefore, to apply a molecular diagnostic method to a complex sample such as stool, a series of pretreatment processes of collecting the complex sample 1 using the complex sample collector 30 and extracting the same with the elution solution E is automated and integrated in a cartridge format, so that the efficiency of sample extraction may be increased, the time and cost involved in sample extraction may be significantly reduced, and precise, uniform, and highly reliable sample pretreatment processes may be conducted regardless of the operator's skill level or working environment. Also, sample contamination or leakage during the process may be fundamentally prevented, and the application to an on-site rapid diagnostic kit is possible, enabling quick achievement of test results within minutes or hours on-site.

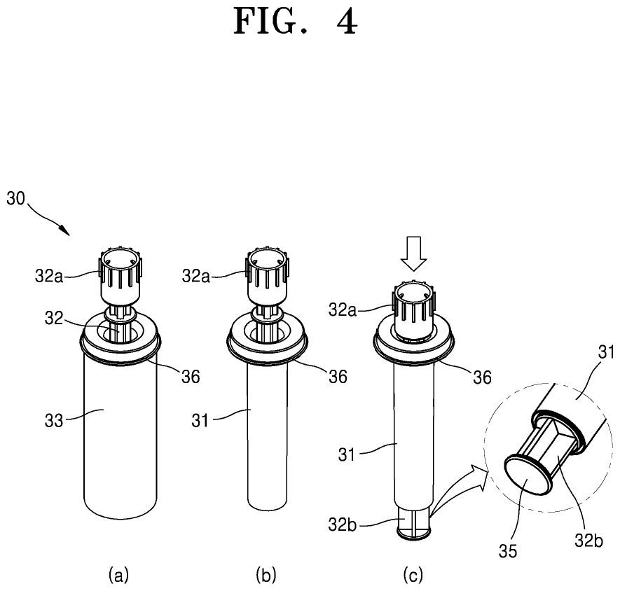

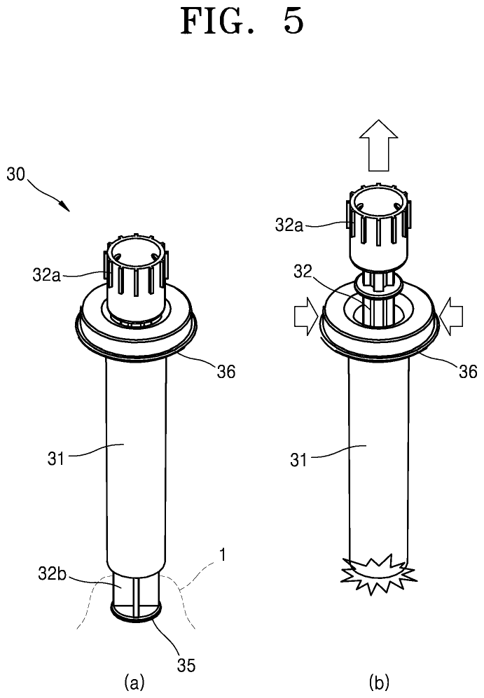

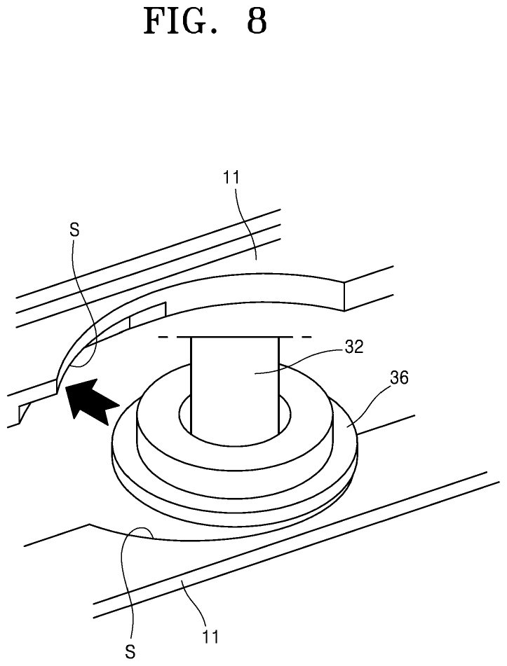

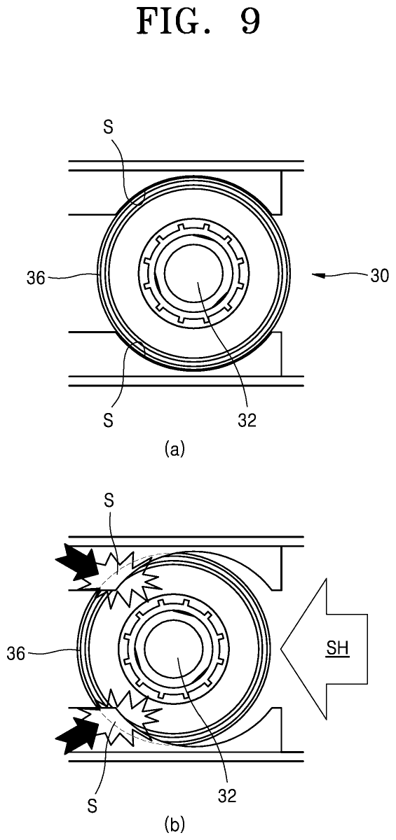

FIGS. 4 to 22 are cross-sectional views of stages of a sample extraction process of the cartridge system 100 for extraction of a complex sample of FIG. 1.

As shown in FIGS. 4 to 22, a more detailed description of the extraction process of the cartridge system 100 for extraction of a complex sample according to some embodiments of the present invention is provided. Firstly, as shown in FIG. 4, using the complex sample collector 30, a complex sample 1 such as stool may be collected and stored.

Here, as shown in FIG. 4, the complex sample collector 30 may include a collector body 31 having the overall shape of a pipe and a hollow portion formed therein and a collection plunger 32 installed to move up and down in the hollow portion, exposing the collecting unit 32b when the button unit 32a is pressed, and sealing the collected complex sample 1 by retracting the collecting unit 32b into the collector body 31 when the button unit 32a is raised. Also, the complex sample collector 30 may further include a storage container 33 that surrounds and protects at least a portion of the collector body 31 and the collection plunger 32.

Therefore, as shown in (a) of FIG. 4, a collector may store the complex sample collector 30 using the storage container 33 during regular times. Also, during collection preparation, the collector may separate the storage container 33 as shown in (b) of FIG. 4, and expose the collecting unit 32b externally by pressing the button unit 32a as shown in (c) of FIG. 4.

Subsequently, as shown in (a) of FIG. 5, the collector may insert the exposed collecting unit 32b into the complex sample 1 and evenly apply the sample to the inner surface of the collecting unit 32b. Following this, as shown in (b) of FIG. 5 the collector may lift the button unit 32a to seal the collecting unit 32b inside the collector body 31.

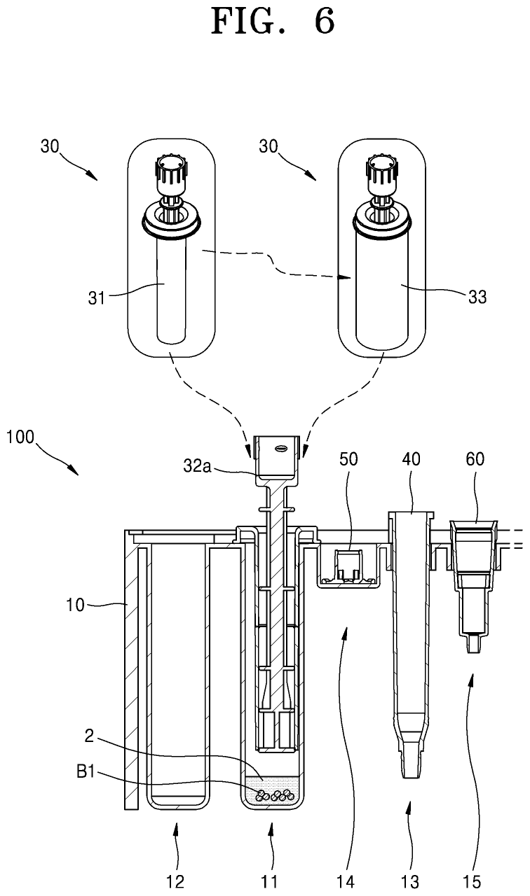

Next, as shown in FIG. 6, the collector may insert the collector body 31 directly into the buffer solution receiving unit 11 or temporarily store it in the storage container 33. Then, after separating the storage container 33, the collector may insert the collector body 31 into the buffer solution receiving unit 11 to immerse the collecting unit 32b of the complex sample collector 30, which has collected the complex sample 1, such as stool, in the buffer solution 2.

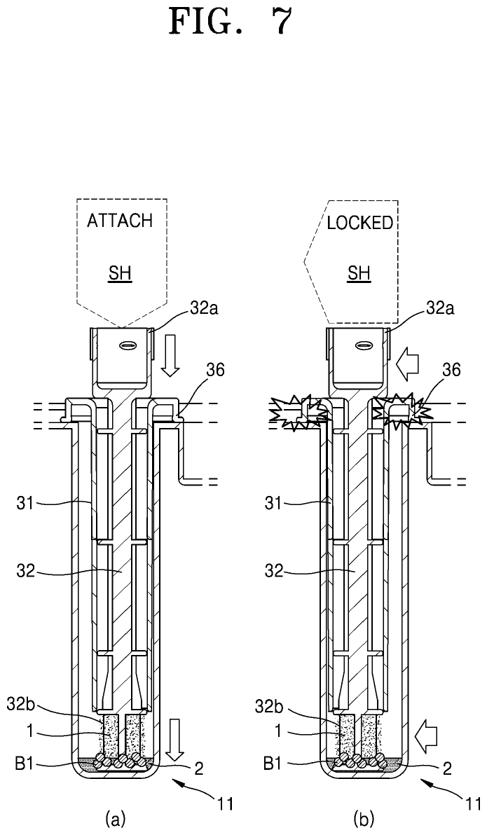

In this case, as shown in FIGS. 7 to 9, the complex sample collector 30 may further include a locking flange unit 36 installed at the entrance of the collector body 31 and shaped to correspond to a height-lock slot S formed on the upper surface of the buffer solution receiving unit 11 such that the spin head SH of the robot device can be inserted in the direction of the height-lock slot S and locked into the height-lock slot S. The complex sample collector 30 may be inserted and locked into the height-lock slot S using the locking flange unit 36.

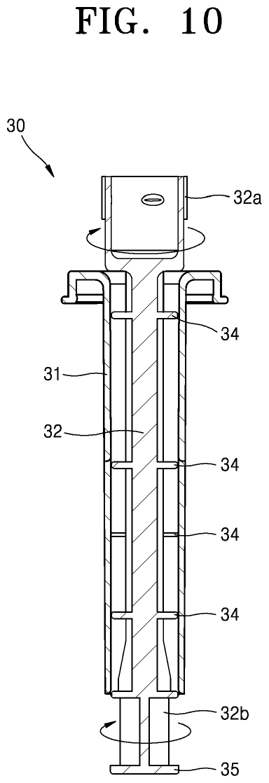

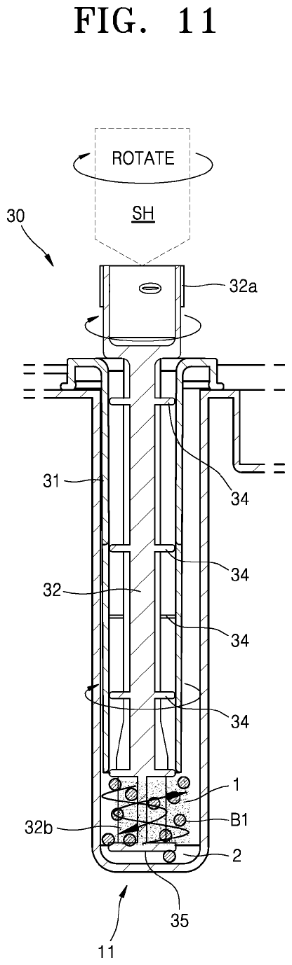

Next, as shown in FIGS. 10 and 11, the complex sample collector 30 may further include an isolation protrusion unit 34 formed on the side surface of the collection plunger 32 and spaced apart from the collector body 31 to facilitate the rotation of the collection plunger 32 when the button unit 32a descends, and a forced-engagement protrusion unit 35 formed on the side surface of the collection plunger 32 to be forcibly engaged with the collector body 31 such that the collecting unit 32b is sealed when the button unit 32a rises. When the button unit 32a descends, the spin head SH of the robot device may freely rotate the collecting unit 32b, allowing the complex sample 1 to mix with the buffer solution 2 or the mixed beads B1 included in the buffer solution 2.

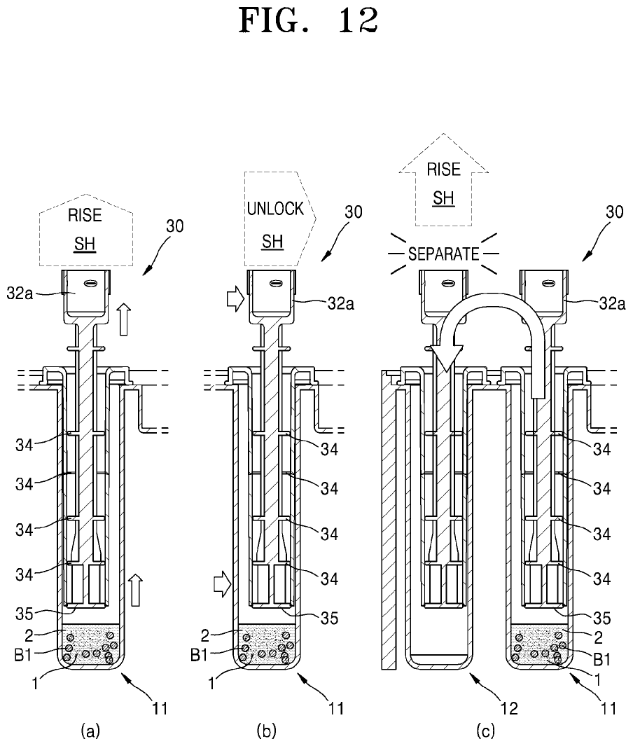

Subsequently, as shown in (a) of FIG. 12, the spin head SH of the robot device raises the button unit 32a to seal the collecting unit 32b. Then, as shown in (b) of FIG. 12, the position of the spin head SH is moved to the right to release the height lock, and finally, as shown in (c) of FIG. 12, the complex sample collector 30 may be temporarily stored in the complex sample collector disposal unit 12 for subsequent disposal of the complex sample collector 30.

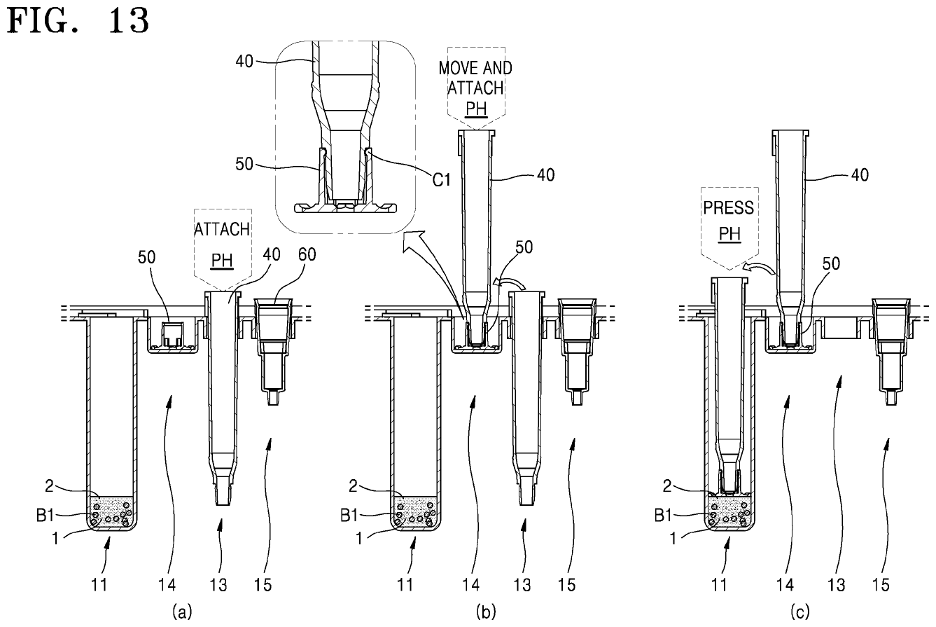

Next, as shown in (a) of FIG. 13, the pump head PH of the robot device is equipped with the first tip 40, and as shown in (b) of FIG. 13, the filter unit 50 may be attached to the first tip 40.

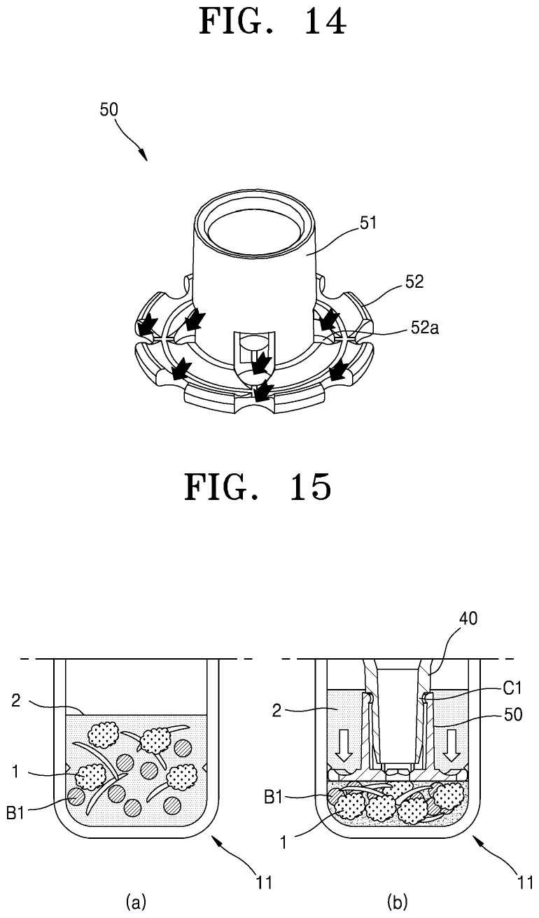

Here, as shown in FIG. 14, the filter unit 50 may include a filter unit body 51 forcibly engaged with the first tip 40 and a flange unit 52 formed at the bottom of the filter unit body 51 and having multiple filter holes 52a formed to primarily filter, for example, fibers or debris contained in the buffer solution 2.

Subsequently, as shown in (a) of FIG. 15, relatively large debris, such as fibers or debris, in the buffer solution 2 inside the buffer solution receiving unit 11 may be pressed through the filter unit 50 as shown in (b) of FIG. 15, allowing the fibers or debris to be primarily filtered, thereby positioning the buffer solution 2 in the upper part of the buffer solution receiving unit 11. Consequently, the liquid substance or solid substance of the complex sample 1 may be dissolved in the buffer solution 2 and positioned in the upper part by passing through the filter unit 50.

Here, as shown in (b) of FIG. 15, the first tip 40 may include a first forced-engaging unit C1 formed to forcibly engage with the filter unit 50, and the buffer solution receiving unit 11 may include a second forced-engaging step unit C2 formed to forcibly engage with the filter unit 50. A first engagement strength of the first forced-engaging unit C1 may be lower than a second engagement strength of the second forced-engaging step unit C2 such that, after the primary filtering by the filter unit 50, the filter unit 50 remains in the buffer solution receiving unit 11, and instead, the first tip 40 may be easily separated from the filter unit 50.

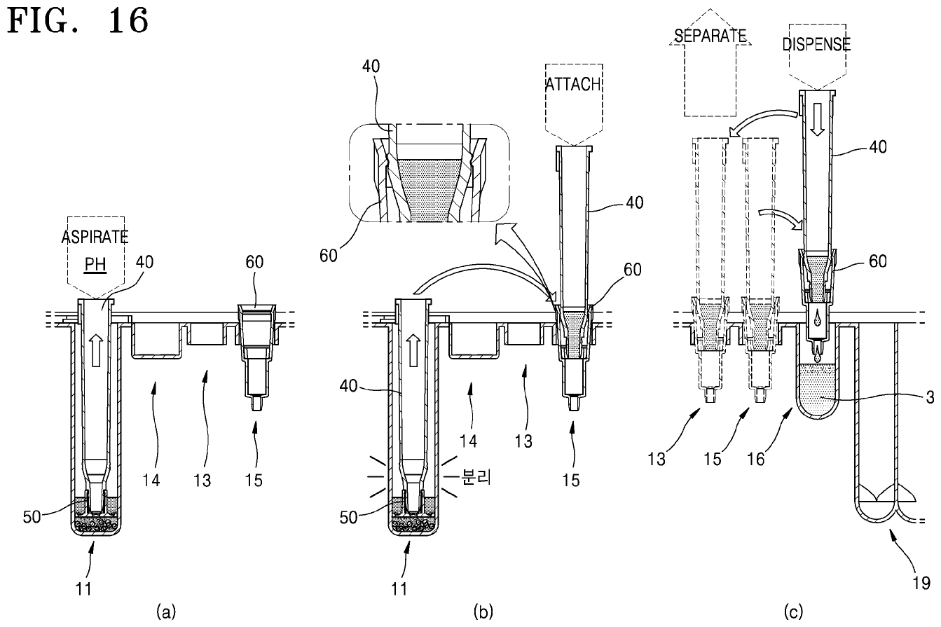

Therefore, as shown in (a) of FIG. 16, the pump head PH of the robot device may aspirate the buffer solution 2 using the first tip 40, and the first tip 40 may rise to separate from the filter unit 50 such that the filter unit 50 remains in the buffer solution receiving unit 11. This separation may be achieved by the difference between the first engagement strength and the second engagement strength. The buffer solution 2 aspirated into the first tip 40 may contain the liquid substance of the complex sample 1 or the solid substance of the complex sample 1 dissolved in the buffer solution 2.

Next, as shown in (b) of FIG. 16, the second tip 60 may be attached to the first tip 40, and as shown in (c) of FIG. 16, the pump head PH of the robot device may use the second tip 60 to dispense the sample solution 3 to the sample solution receiving unit 16 while secondarily filtering the buffer solution 2. After dispensing the sample solution 3, the remaining first tip 40 and the attached second tip 60 may be temporarily stored in the buffer solution receiving unit 11, the first tip receiving unit 13, or the second tip receiving unit 15 for subsequent disposal.

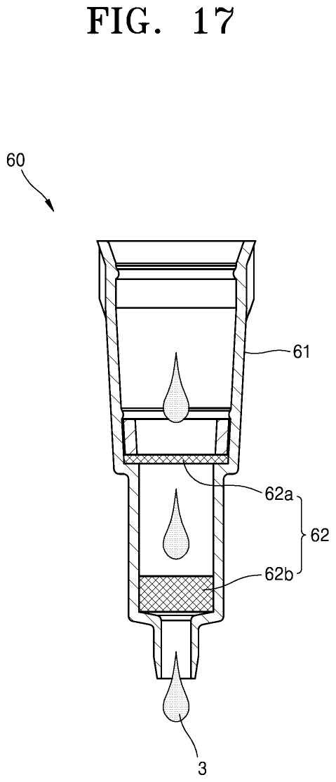

Here, for example, as shown in FIG. 17, the second tip 60 may include a second tip body 61 forcibly engaged with the first tip 40 and a secondary filter 62 installed inside the second tip body 61 and including a mesh filter 62a or a membrane filter 62b, and may secondarily filter debris or fibers that may remain in the buffer solution 2.

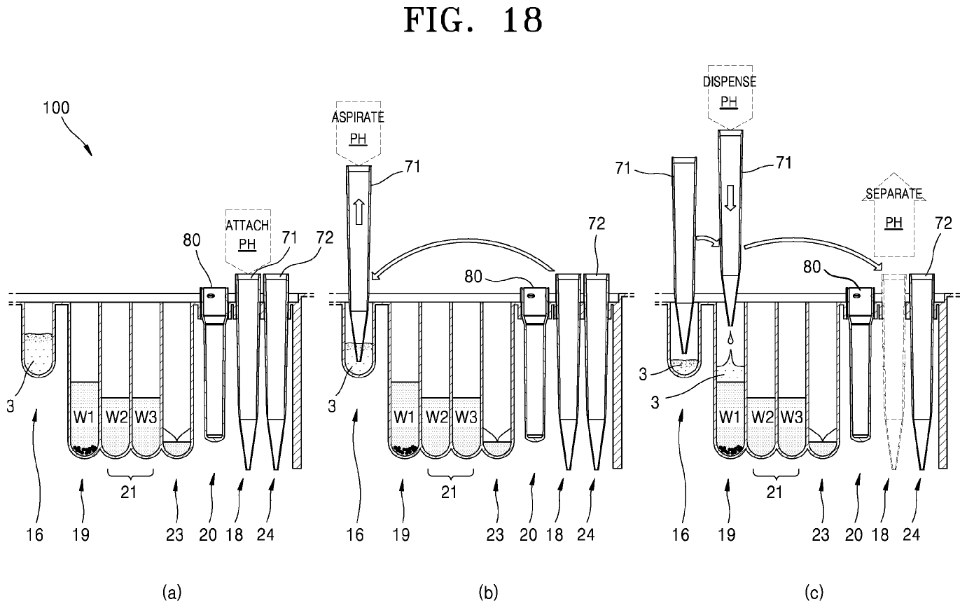

Subsequently, as shown in (a) of FIG. 18. The pump head PH of the robot device may be coupled with the first precision dispensing tip 71, and as shown in (b) of FIG. 18, it may aspirate the sample solution 3 from the sample solution receiving unit 16. Subsequently, as shown in (c) of FIG. 18, the pump head PH of the robot device may dispense the aspirated sample solution 3 into the first washing chamber unit 19 such that the sample solution 3 is mixed with the first washing solution W1 and the magnetic beads B2 included in the first washing solution W1, allowing only the DNA components to remain on the magnetic beads B2.

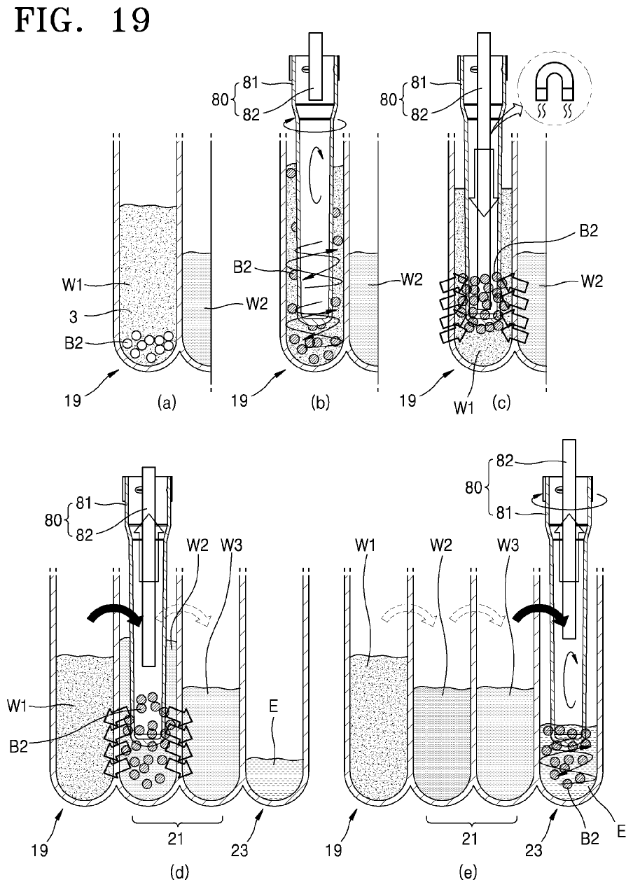

Here, as shown in (a) of FIG. 19, the first washing solution W1 mixed with the sample solution 3 may contain a plurality of magnetic beads B2 therein.

Next, as shown in (b) and (c) of FIG. 19, the spin head SH of the robot device may be coupled with the stirring tip 80 and may rotate or move up and down to stir the magnetic beads B2 in the first washing chamber unit 19 with the first washing solution W1. Additionally, the magnetic beads B2 may be magnetically attached to the stirring tip 80.

Subsequently, as shown in (d) and (e) of FIG. 19, in N Nth washing chamber units (N is a natural number greater than or equal to 2), the magnetic beads B2 may be repeatedly stirred N times with the Nth washing solutions WN and W3 as the stirring tip 80 of the spin head SH of the robot device rotates or moves up and down, so that only the DNA component remains.

Here, as shown in FIG. 19, the stirring tip 80 may include a rotatable stirring tip body 81 having an overall hollow shape and coupled with the spin head SH and a magnetic bar 82 installed inside the stirring tip body 81 and magnetically attaching the magnetic beads B2 to the stirring tip body 81 while moving up and down.

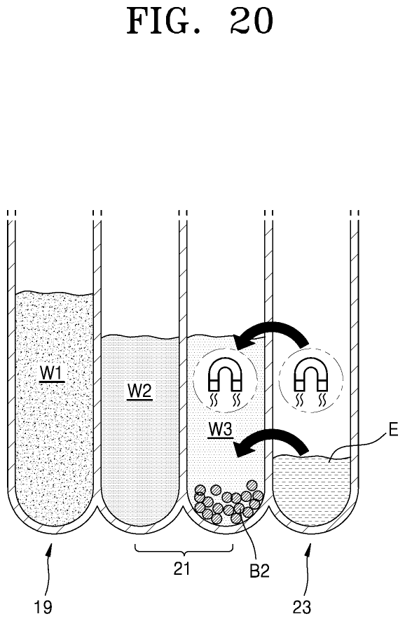

Therefore, as shown in FIG. 20, using the stirring tip 80 allows both the rotation of the stirring tip body 81 and the up-and-down movement of the magnetic bar 82 so that the magnetic beads B2 are shaken and thus can be smoothly stirred. The magnetic beads B2 may be magnetically attached to the stirring tip 80 by the magnetic bar 82 and may be transferred from the first washing chamber unit 19 to the Nth washing chamber unit 21 after stirring, sequentially moved through the Nth washing chamber unit 21, and ultimately transferred to the elution chamber unit 23. Consequently, the elution solution E may be accommodated in the elution chamber unit 23.

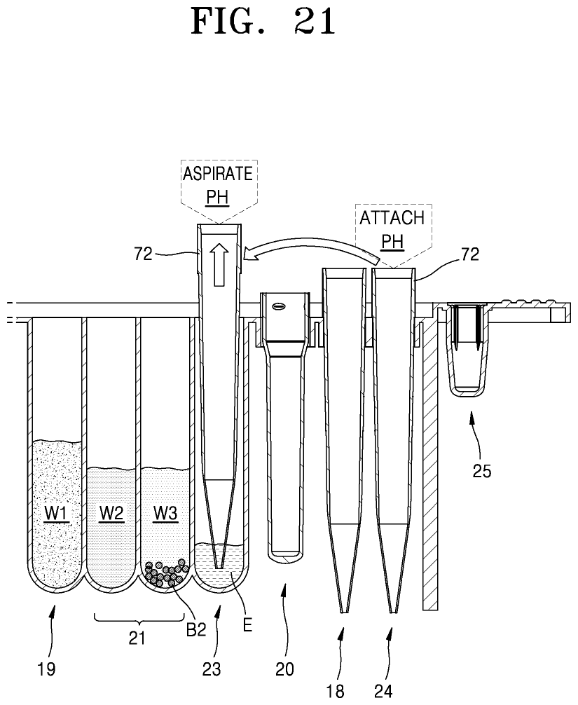

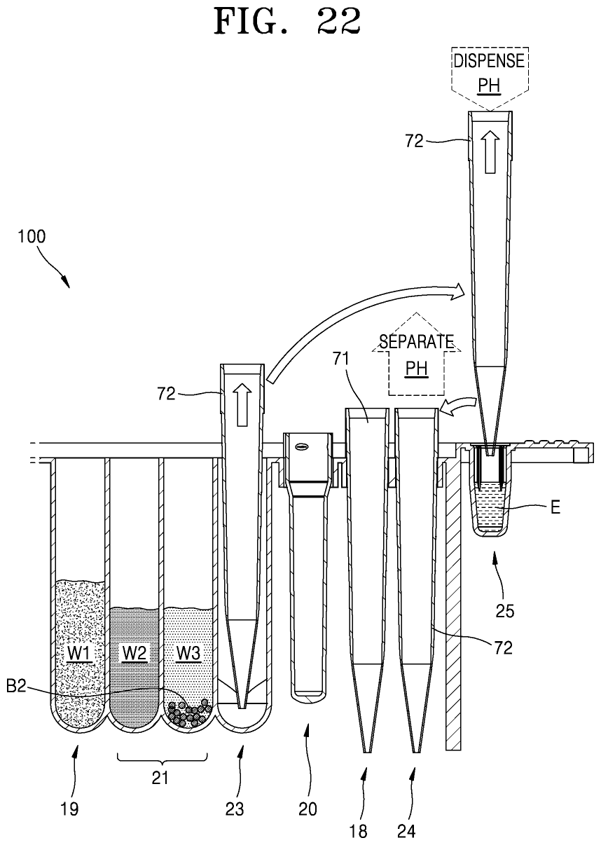

Subsequently, as shown in FIG. 21, the pump head PH of the robot device may be coupled with the second precision dispensing tip 72, and may aspirate the elution solution E accommodated in the elution chamber unit 23 using the second precision dispensing tip 72.

Then, as shown in FIG. 22, the aspirated elution solution E may be dispensed into the pipetting chamber unit 25.

Therefore, the operator may obtain the elution solution E that has undergone the entire pretreatment processes from the pipetting chamber unit 25 and proceed with the analysis process and post-treatment process.

Consequently, molecular diagnostic testing may be performed on the complex sample 1, such as the stool of an examination subject, consisting of a mixture of liquid and solid phases, so that physical discomfort or resistance in the examination subject can be prevented and easy sample collection significantly increases the precision of test results, while minimizing the spread of bacteria or viruses during the sample collection process.

However, this series of complex sample extraction processes is not limited to the drawings and modifications and changes can be made by those skilled in the art without departing from the technical spirit of the present invention.

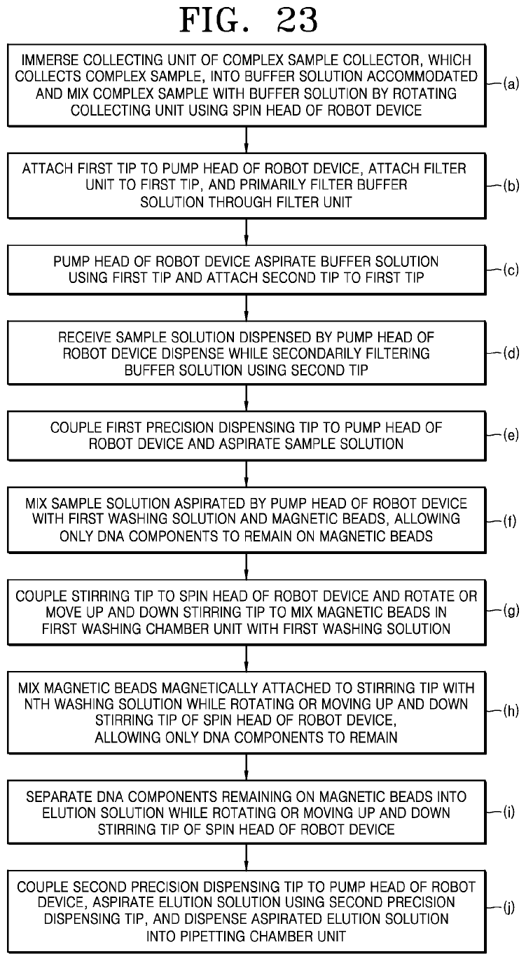

FIG. 23 is a flowchart illustrating a complex sample extracting method according to some embodiments of the present invention.

As shown in FIGS. 1 to 23, a complex sample extracting method according to some embodiments of the present invention may include: (a) immersing the collecting unit 32b of the complex sample collector 30, which collects the complex sample 1, into the buffer solution 2 accommodated in the buffer solution receiving unit 11 and mixing the complex sample 1 with the buffer solution 2 or the mixed beads B1 contained in the buffer solution 2 by rotating the collecting unit 32b using the spin head SH of the robot device; (b) attaching the first tip 40 to the pump head PH of the robot device, attaching the filter unit 50 to the first tip 40, and primarily filtering the buffer solution 2 accommodated inside the buffer solution receiving unit 11 through the filter unit 50; (c) at the pump head PH of the robot device, aspirating the buffer solution 2 using the first tip 40 and attaching the second tip to the first tip 40; (d) receiving the sample solution 3 that the pump head PH of the robot device dispenses while secondarily filtering the buffer solution 2 contained in the first tip 40 using the second tip 60; (e) coupling the first precision dispensing tip 71 to the pump head PH of the robot device and aspirating the sample solution 3; (f) mixing the sample solution 3 aspirated by the pump head PH of the robot device with the first washing solution W1 accommodated in the first washing chamber unit 19 and the magnetic beads B2 included in the first washing solution W1, allowing only DNA components to remain on the magnetic beads B2; (g) coupling the stirring tip 80 to the spin head SH of the robot device and rotating or moving up and down the stirring tip 80 to mix the magnetic beads B2 in the first washing chamber unit 19 with the first washing solution W1, allowing only the DNA components to remain; (i) separating the DNA components remaining on the magnetic beads B2 into the elution solution E while rotating or moving up and down the stirring tip 80 of the spin head SH of the robot device; and (j) coupling the second precision dispensing tip 72 to the pump head PH of the robot device, aspirating the elution solution E using the second precision dispensing tip 72, and dispensing the aspirated elution solution E into the pipetting chamber unit 25.

The complex sample extracting method may further include, before or after operation (i), (h) mixing the magnetic beads B2 with the Nth washing solution WN (N is a natural number greater than or equal to 2) while rotating or moving up and down the stirring tip 80 of the spin head SH of the robot device, allowing only the DNA components to remain.

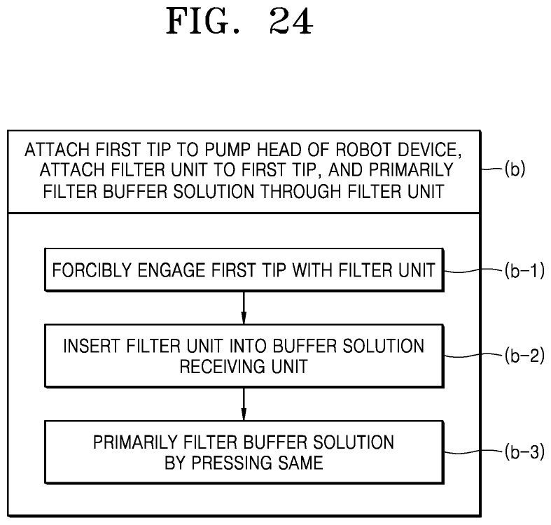

FIG. 24 is a flowchart illustrating in more detail operation (b) of the complex sample extracting method of FIG. 23.

As shown in FIGS. 1 to 24, operation (b) may include: (b-1) forcibly engaging the first tip 40 with the filter unit 50; (b-2) inserting the filter unit 50 into the buffer solution receiving unit 11; and (b-3) primarily filtering the buffer solution 2 by pressing the buffer solution 2 mixed with the complex sample 1 using the filter unit 50.

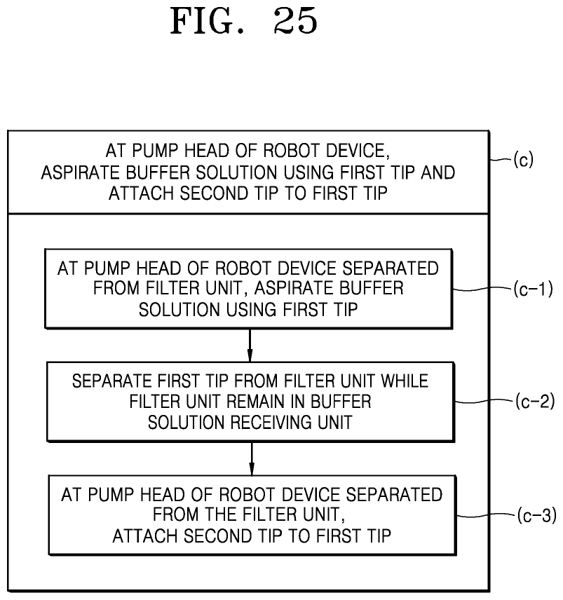

FIG. 25 is a flowchart illustrating in more detail operation (c) of the complex sample extracting method of FIG. 23.

As shown in FIGS. 1 to 25, operation (c) may include: (c-1) at the pump head PH of the robot device separated from the filter unit 50, aspirating the buffer solution 2 using the first tip 40; (c-2) raising the pump head PH of the robot device to separate the first tip 40 from the filter unit 50 while being attached to the pump head PH of the robot device while the filter unit 50 remains in the buffer solution receiving unit 11; and (c-3) at the pump head PH of the robot device separated from the filter unit 50, attaching the second tip 60 to the first tip 40.

While the present invention has been particularly shown and described with reference to embodiments thereof, it will be understood by those of ordinary skill in the art that various changes in form and details may be made therein without departing from the spirit and scope of the present invention as defined by the following claims. Therefore, the scope of the present invention should be defined only by the appended claims.

1. A cartridge system for extraction of a complex sample, comprising:
a cartridge body;
a complex sample collector for collecting a complex sample to provide same to the cartridge body;
a buffer solution receiving unit formed in the cartridge body and configured to accommodate a buffer solution therein and provide a space in which, after a collecting unit of the complex sample collector is immersed in the buffer solution, the collecting unit is rotated by a spin head of a robot device such that the complex sample is mixed with the buffer solution or mixed beads included in the buffer solution; and
a complex sample collector disposal unit formed in the cartridge body and configured to temporarily store the complex sample collector to discard same after the complex sample is provided to the buffer solution receiving unit.

2. The cartridge system of claim 1, wherein the complex sample collector comprises:
a collector body having an overall shape of a pipe and a hollow portion formed therein; and
a collection plunger installed to move up and down in the hollow portion, exposing the collecting unit when the button unit is pressed, and sealing the collected complex sample by retracting the collecting unit into the collector body when the button unit is raised after collecting the complex sample.

3. The cartridge system of claim 2, wherein the complex sample collector further comprises:
an isolation protrusion unit formed on a side surface of the collection plunger and spaced apart from the collector body to facilitate rotation of the collection plunger when the button unit descends; and
a forced-engagement protrusion unit formed on a side surface of the collection plunger to be forcibly engaged with the collector body such that the collecting unit is sealed when the button unit rises.

4. The cartridge system of claim 2, wherein the complex sample collector further comprises a locking flange unit installed at an entrance of the collector body and shaped to correspond to a height-lock slot formed on an upper surface of the buffer solution receiving unit such that the spin head of the robot device can be inserted in the direction of the height-lock slot and locked into the height-lock slot.

5. The cartridge system of claim 1, further comprising:
a first tip receiving unit formed in the cartridge body and configured to accommodate the first tip such that a first tip is attached to a pump head of the robot device; and
a filter unit receiving unit formed in the cartridge body and configured to accommodate a filter unit such that the pump head of the robot device attaches the filter unit to the first tip and primarily filters the buffer solution accommodated in the buffer solution receiving unit through the filter unit.

6. The cartridge system of claim 5, further comprising a second tip receiving unit formed in the cartridge body and configured to accommodate a second tip such that the pump head of the robot device uses the first tip to aspirate the buffer solution and attaches the second tip to the first tip.

7. The cartridge system of claim 5, wherein the filter unit comprises:
a filter unit body forcibly engaged with the first tip; and
a flange unit formed on a bottom of the filter unit body and having a plurality of filter holes formed to primarily filter the buffer solution.

8. The cartridge system of claim 5, wherein:
the first tip comprises a first forced-engaging unit formed to forcibly engage with the filter unit,
the buffer solution receiving unit comprises a second forced-engaging step unit formed to forcibly engage with the filter unit, and
a first engagement strength of the first forced-engaging unit is lower than a second engagement strength of the second forced-engaging step unit such that, after the primary filtering by the filter unit, the filter unit remains in the buffer solution receiving unit, and instead, the first tip is easily separated from the filter unit.

9. The cartridge system of claim 5, wherein the second tip further comprises:
a second tip body forcibly engaged with the first tip; and
a secondary filter installed inside the second tip body and comprising a mesh filter or a membrane filter.

10. The cartridge system of claim 5, further comprising:
a sample solution receiving unit formed in the cartridge body and configured to accommodate a sample solution that the pump head of the robot device dispenses while secondarily filtering the buffer solution using the second tip;
a first precision dispensing tip receiving unit formed in the cartridge body and configured to accommodate first precision dispensing tip such that the pump head of the robot device is coupled with the first precision dispensing tip to aspirate the sample solution;
a first washing chamber unit formed in the cartridge body and configured to accommodate a first washing solution and magnetic beads included in the first washing solution such that the sample solution aspirated by the pump head of the robot device is mixed with the first washing solution and the magnetic beads, allowing only DNA components to remain on the magnetic beads;
a stirring tip receiving unit formed in the cartridge body and configured to accommodate a stirring tip such that the spin head of the robot device is coupled with the stirring tip and stirs the magnetic beads in the first washing chamber unit with the first washing solution while rotating or moving up and down; and
an Nth washing chamber unit (N is a natural number greater than or equal to 2) formed in the cartridge body and configured to accommodate an Nth washing solution such that, as the stirring tip of the spin head of the robot device rotates or moves up and down, the magnetic beads are mixed with the Nth washing solution, allowing only DNA components to remain.

11. The cartridge system of claim 10, wherein the stirring tip comprises:
a rotatable stirring tip body having an overall hollow shape and coupled with the spin head; and
a magnetic bar installed inside the stirring tip body and magnetically attaching the magnetic beads to the stirring tip body while moving up and down.

12. The cartridge system of claim 10, further comprising:
an elution chamber unit formed in the cartridge body and configured to accommodate an elution solution such that the DNA components remaining on the magnetic bead are separated into the elution solution as the stirring tip of the spin head of the robot device rotates or moves up and down;
a second precision dispensing tip receiving unit formed in the cartridge body and configured to accommodate a second precision dispensing tip such that the pump head of the robot device attaches to the second precision dispensing tip; and
a pipetting chamber unit formed in the cartridge body and configured to accommodate the elution solution dispensed such that the pump head of the robot device aspirates the elution solution using the second precision dispensing tip and dispenses the aspirated elution solution into the pipetting chamber unit.

13. A complex sample extracting method comprising:
(a) immersing a collecting unit of a complex sample collector, which collects a complex sample, into a buffer solution accommodated in a buffer solution receiving unit and mixing the complex sample with the buffer solution or mixed beads included in the buffer solution by rotating the collecting unit using a spin head of a robot device;
(b) attaching a first tip to a pump head of the robot device, attaching a filter unit to the first tip, and primarily filtering the buffer solution accommodated inside the buffer solution receiving unit through the filter unit;
(c) at the pump head of the robot device, aspirating the buffer solution using the first tip and attaching a second tip to the first tip; and
(d) receiving a sample solution that the pump head of the robot device dispenses while secondarily filtering the buffer solution accommodated in the first tip using the second tip.

14. The complex sample extracting method of claim 13, wherein operation (b) comprises:
(b-1) forcibly engaging the first tip with the filter unit;
(b-2) inserting the filter unit into the buffer solution receiving unit; and
(b-3) primarily filtering the buffer solution by pressing the buffer solution mixed with the complex sample using the filter unit.

15. The complex sample extracting method of claim 14, wherein operation (c) comprises:
(c-1) at the pump head of the robot device separated from the filter unit, aspirating the buffer solution using the first tip;
(c-2) raising the pump head of the robot device to separate the first tip from the filter unit while being attached to the pump head of the robot device while the filter unit remains in the buffer solution receiving unit; and
(c-3) at the pump head of the robot device separated from the filter unit, attaching the second tip to the first tip.

16. The complex sample extracting method of claim 13, further comprising:
(e) coupling a first precision dispensing tip to the pump head of the robot device and aspirating the sample solution;
(f) mixing the sample solution aspirated by the pump head of the robot device with a first washing solution accommodated in a first washing chamber unit and magnetic beads included in the first washing solution, allowing only DNA components to remain on the magnetic beads;
(g) coupling a stirring tip to the spin head of the robot device and rotating or moving up and down the stirring tip to mix the magnetic beads in the first washing chamber unit with the first washing solution, allowing only the DNA components to remain;
(i) separating the DNA components remaining on the magnetic beads into an elution solution while rotating or moving up and down the stirring tip of the spin head of the robot device; and
(j) coupling a second precision dispensing tip to the pump head of the robot device, aspirating the elution solution using the second precision dispensing tip, and dispensing the aspirated elution solution into a pipetting chamber unit.

17. The complex sample extracting method of claim 16, further comprising:
before or after operation (i),
(h) mixing the magnetic beads with an Nth washing solution (N is a natural number greater than or equal to 2) while rotating or moving up and down the stirring tip of the spin head of the robot device, allowing only the DNA components to remain.
